(19) United States
(12) Patent Application Publication
(10) Pub. No.: US 2026/0207855 A1
(43) Pub. Date: Jul. 23, 2026

(54) INJECTION NEEDLE APPARATUS WITH SAFE-PROTECTION FUNCTION

(71) Applicant: YAO SHENG ELECTRONIC CO., LTD., Taoyuan City (TW)

(72) Inventors: SHAN-PU YU, NEW TAIPEI CITY (TW); HUO-HSI SU, NEW TAIPEI CITY (TW)

(73) Assignee: YAO SHENG ELECTRONIC CO., LTD., Taoyuan City, TW (TW)

(21) Appl. No.: 19/568,748

(22) Filed: Mar. 16, 2026

Related U.S. Application Data
(63) Continuation of application No. 18/206,099, filed on Jun. 6, 2023.

(51) Int. Cl. A61M 5/32; A61M 5/315; A61M 5/34

(52) U.S. Cl. CPC A61M 5/3221; A61M 5/3204; A61M 5/347; A61M 5/31501

(57) ABSTRACT

An injection needle apparatus with safe-protection function includes: an injection module having an injection cylinder and a pullable piston rod; a needle module including a needle seat and an injection needle; the needle seat having a seat body and a buckle. In installation state, a safety sleeve covering movably around the injection cylinder between an installation position and a protection position. In the installing position, a first end portion of the safety sleeve is near the buckle and the second end portion thereof is near the handhold portion; while in the protection position, the first end portion is protruded from the injection cylinder for covering the injection needle and the second end portion is near the head portion of the injection cylinder. An inner side of the sleeve body is formed with a first and a second annular flange; and an annular groove is formed therebetween for confining the buckle.

The invention is a continuation in part (CIP) of the U.S. patent application Ser. No. 18/206,099 filed at Jun. 6, 2023, invented and assigned to the inventor of the present invention, and thus the contents of the U.S. patent application Ser. No. 18/206,099 is incorporated into the present invention as a part of the specification.

### FIELD OF THE INVENTION

The present invention is related to injection needles, and in particular to an injection needle apparatus with safe-protection function, in that as disposal of the injection cylinder, a set of needle seat and safety sleeve can be left from the needle seat separately.

### BACKGROUND OF THE INVENTION

Recently, to avoid injection needle to hurt medical members or cleaning members so as to induce diseases or infections to these members, safety injection devices are developed so that after the injection cylinders are used, need heads can be isolated for preventing the people from being hurt due to contact the injection cylinder.

In current injection needle, the injection needle is fixed to the injection cylinder. In use, a safety sleeve sleeves the injection needle to avoid to hurt people. Since the injection needle and the safety sleeve are connected to the front end of the injection cylinder, when an injection needle is disposed, it has a large volume which induces difficulty in waste disposal and the risk of un-safety. Furthermore, an injection needle is fixed to the injection cylinder, they being not separable. As a result, the injection cylinder cannot inject drug liquid into a fixed injection device, such as a drip cylinder.

### SUMMARY OF THE INVENTION

Accordingly, the object of the present invention is to provide a injection needle apparatus with safe-protection function, in that the structure of the needle seat 21 and the safety sleeve 3 is simple and can be assembled and operated easily. Furthermore, the structure of needle seat 21 and safety sleeve 3 can be separated with the injection cylinder 11 so that undesired waste can be disposed easily. Furthermore, the present invention can be applied in injection of drug liquid.

To achieve above object, the present invention provides a injection needle apparatus with safe-protection function, comprising: an injection module including an injection cylinder and a pullable piston rod installed within the injection cylinder; the injection cylinder including a main body, a head portion and a handhold portion; the head portion and the handhold portion being at two ends of the main body, respectively; a needle module including a needle seat and an injection needle; the needle seat having a seat body and a buckle; in installation state, the seat body covering the head portion and holding the injection needle; the buckle being integrally connected to the seat body at an end near the main body and around the seat body; a safety sleeve (3) covering movably around the injection cylinder (11) between an installation position and a protection position; the safety sleeve having a sleeve body, a first end portion, and a second end portion; the first end portion and the second end portion being at two opposite ends of the sleeve body; wherein in the installing position, the first end portion is near the buckle and the second end portion is near the handhold portion; while in the protection position, the first end portion is protruded from the injection cylinder for covering the injection needle and the second end portion is near the head portion; and an inner side of the sleeve body (31) near the second end portion being formed with a buckling portion; the buckling portion being used to buckle with the buckle (212) so that the safety sleeve (3) and the needle seat (21) are fixed together for separating from the injection cylinder and after separation, the safety sleeve covers the needle seat.

A fixing structure (50) is formed at a lower end of the injection cylinder (11) near the handhold portion (113) and a rear end of the safety sleeve (3) so that when the safety sleeve retracts backwards to be near the handhold portion (113) of the injection cylinder (11), the safety sleeve can be fixed as the position to prevent the safety sleeve from moving with the injection needle.

A tight buckling structure (52) is formed at the head portion (112) of the injection cylinder (11) and a rear end of the seat body (211) of the needle seat (21); therefore, the seat body (211) of the needle seat (21) can be tightly fixed to the head portion (112) of the needle seat (21) to prevent the needle seat (21) from separating with the head portion (112) of the injection cylinder (11).

The buckling portion includes a first annular flange (311) and a second annular flange (312); an annular groove (313) being formed between the first annular flange and the annular second flange; the annular groove being used to confine the buckle.

The various objects and advantages of the present invention will be more readily understood from the following detailed description when read in conjunction with the appended drawing.

### BRIEF DESCRIPTION OF THE DRAWINGS

FIG. 1 is a cross sectional view of the injection needle apparatus with safe-protection function of the present invention, which is in an unused state.

FIG. 2 is a perspective view showing the injection cylinder in the embodiment of FIG. 1 according to the present invention.

FIG. 3 is a perspective view showing the needle seat of the present invention based on the embodiment shown in FIG. 1.

FIG. 4 is a backside view of the needle seat in the embodiment of FIG. 1.

FIG. 5 is a cross section view of the needle seat of the present invention.

FIG. 6 is a schematic view showing a partial part of the safety sleeve of the present invention.

FIG. 7 is a cross section view showing that the safety sleeve of the present invention is in a protection state.

FIG. 8 is a cross section view showing the safety sleeve of the present invention, where the safety sleeve is fixed to the needle seat and does not separate with the injection cylinder.

FIG. 9 is a cross section view of the present invention, where the safety sleeve is fixed to the needle seat, while being separate with the injection cylinder.

FIG. 10 is a perspective view of the second embodiment of the present invention, where the fixing structure is a partial annular protrusion.

FIG. 11 is a perspective view of the second embodiment of the present invention, where the fixing structure is a complete annular protrusion.

FIG. 12 is a cross section view of the third embodiment of the present invention.

FIG. 13 is a perspective view of the fourth embodiment of the present invention.

FIG. 14 is a perspective view showing the arrangement of the injection cylinder and the needle seat.

FIG. 15 is a perspective view about another arrangement of the injection cylinder and the needle seat.

FIG. 16 is a perspective view showing a further arrangement of the injection cylinder and the needle seat.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

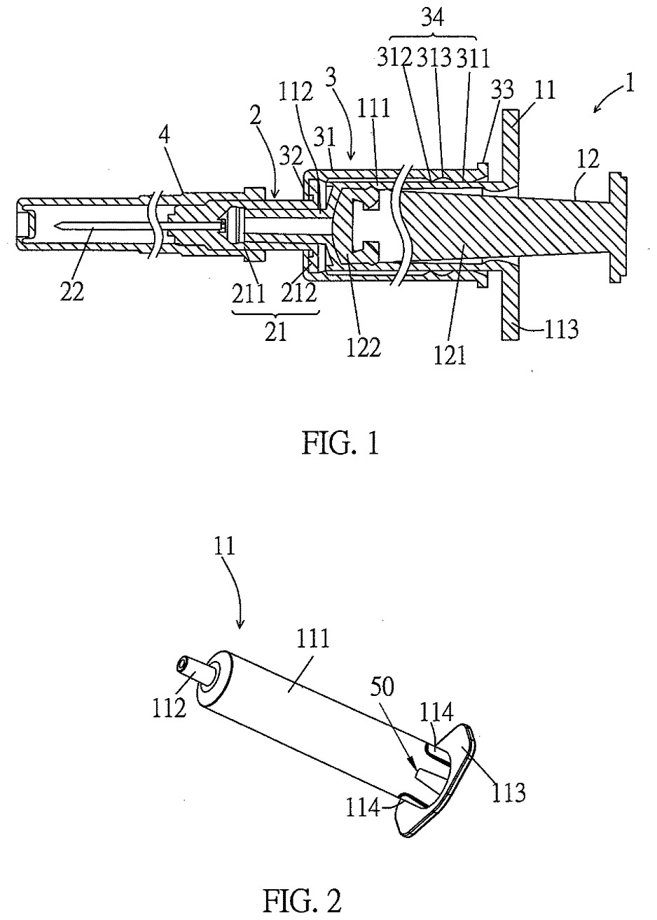

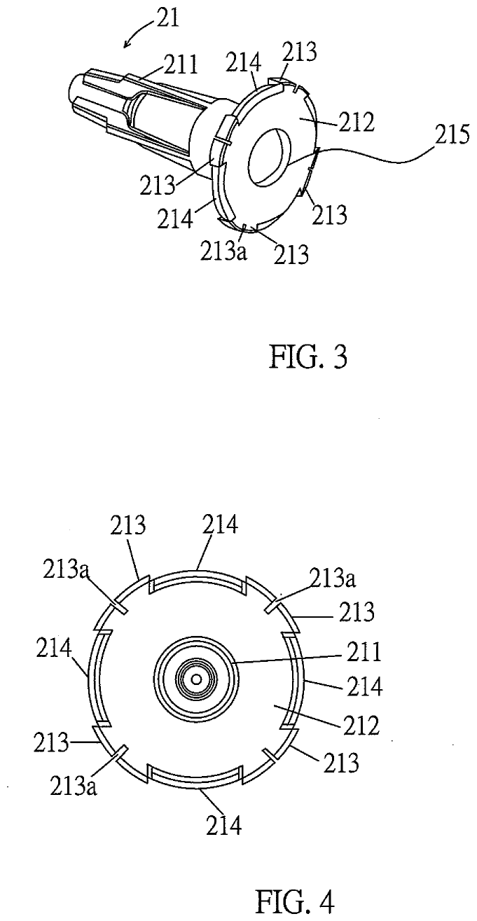

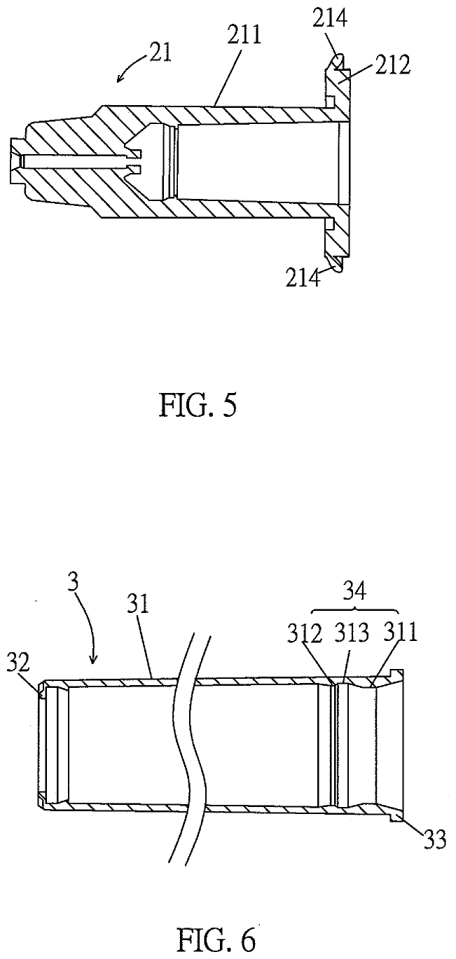

Referring to FIG. 1, the structure of the injection needle apparatus with safe-protection function according to the present invention is illustrated. The present invention includes the following elements with reference to FIGS. 1, 3 and 5.

An injection module 1 includes an injection cylinder 11 and a pullable piston rod 12 installed within the injection cylinder 11. The injection cylinder 11 includes a main body 111, a head portion 112 and an extended handhold portion 113 at two ends of the main body 111, respectively. The piston rod 12 has a rod portion 121 and a piston 122 connected at a front end of the rod portion 121.

Referring to FIGS. 1, 3 and 5, a needle module 2 includes a needle seat 21 and an injection needle 22. The needle seat 21 has a seat body 211 and a buckle 212. In installation state, the seat body 211 covers the head portion 112 and holding the injection needle 22. The buckle 212 is integrally formed with the seat body 211 at an end near the main body 11 and around the seat body 211.

In this embodiment, the buckle 212 is extended with a plurality of buckling blocks 213 around an outer periphery thereof and a plurality of thin portions 214. In FIG. 3, four buckling blocks 213 and four thin portions 214 which are alternatively arranged are shown. In this embodiment, each thin portion 214 is realized by a thin arc so that the buckle 212 can be elastically reduced inwards along a radial direction. That is, the buckle 212 is soft. Each buckling block 213 is formed with at least one recess 213a.

Referring to FIGS. 14 to 16, it is illustrated that a tight buckling structure 52 is formed at the head portion 112 of the injection cylinder 11 and a rear end of the seat body 211 of the needle seat 21. Therefore, when a medical member uses the injection needle of the present invention, the seat body 211 of the needle seat 21 can be tightly fixed to the head portion 112 of the needle seat 21 to prevent the needle seat 21 from separating with the head portion 112 of the injection cylinder 11 due to a mistake of the operation.

Referring to FIG. 14, it is illustrated the first embodiment of the tight buckling structure of the present invention, in that the head portion 112 of the injection cylinder 11 is formed as a tapered portion 521. A cross sections in radial direction becomes larger and larger from a distal end to the end near the extended handhold portion 113. A maximum radial length in the cross sections of the tapered portion 521 is larger than a maximum radial length of an opening 215 of the seat body 211 so that when the needle seat 21 is assembled to the head portion 112, the seat body 211 will buckle the tapered portion 521 of the head portion 112 with the movement of the tapered portion 521 into the opening 215 so that the needle seat 21 is tightly buckled with the head portion 112 and not pulled out easily. Therefore, this asserts the safety in operation of the needle of the present invention.

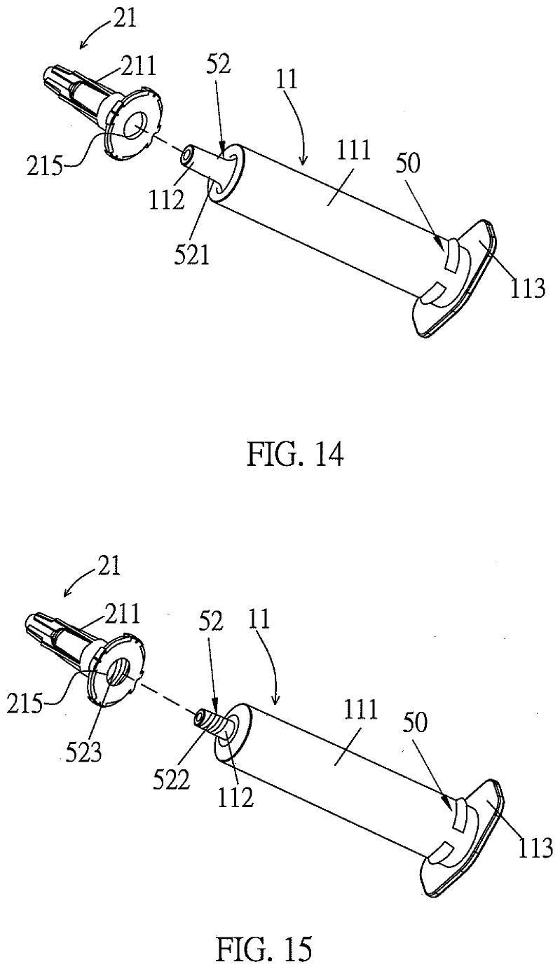

With reference to FIG. 15, the second embodiment about the tight-buckling structure 52 of the present invention is illustrated, wherein an outer surface of the head portion 112 of the injection cylinder 11 is formed with an outer thread 522, and an inner wall of the opening 215 of the needle seat 21 is formed with a respective inner thread 523. When assembling the needle seat 21 into the head portion 112, the seat body 211 can be threaded combined with the head portion 112 so that the needle seat 21 is tightly engaged to the injection cylinder 11.

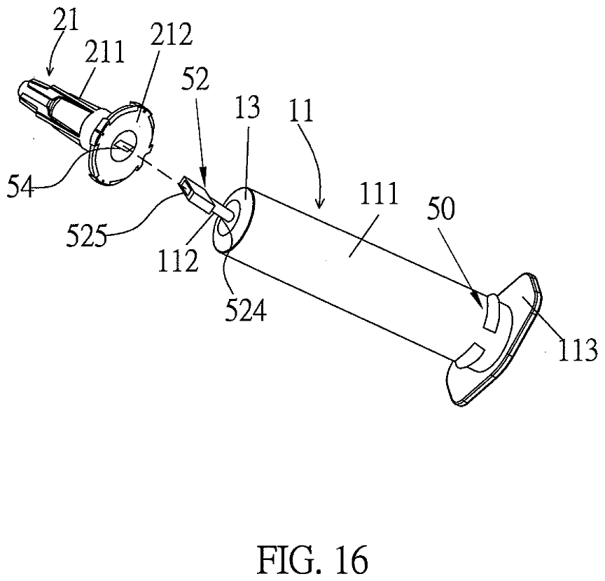

With reference to FIG. 16, the third embodiment of the present invention is illustrated, wherein a front end of the head portion 112 of the injection cylinder 11 is formed with a flat enlarged portion; and the buckle 212 of the needle seat 21 is formed with a respective flat opening 54. A flat enlarged portion of the injection cylinder 11 includes a first portion 525 and a second portion 524 which is connected between the first portion 525 and a front end 13 of the body 111. The first portion 525 could pass through the flat opening 54. A maximum radial width of the first portion 525 is larger than than a minimum radial width of the flat opening 54. Therefore, when the first portion 525 is within an inner side of the flat opening 54, rotating the flat enlarged portion of the injection cylinder 11 will cause the first portion 525 is confined within the flat opening 54. Furthermore, a maximum radial width in cross sections of the second portion 524 is about equal to a minimum radial width of the flat opening 54 so that the whole head portion 112 can be fixed within the needle seat 21 so that the injection cylinder 11 is tightly fixed to the injection cylinder 11.

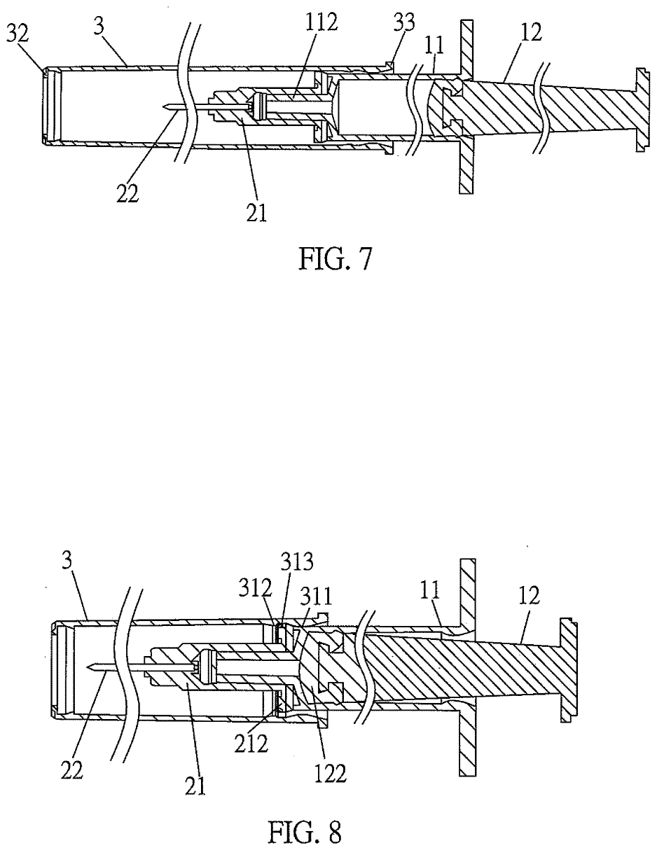

With reference to FIGS. 1, 6 and 7, a safety sleeve 3 sleeves movably around the injection cylinder 11 between an installation position (referring to FIG. 1) and a protection position (referring to FIG. 7). The safety sleeve 3 has a sleeve body 31, a first end portion 32, and a second end portion 33. The first end portion 32 and the second end portion 33 are at two opposite ends of the sleeve body 31. In the installing position, the first end portion 32 being near the buckle 212 and the second end portion 33 being near the handhold portion 113

An inner side of the sleeve body 31 near the second end portion 33 is formed with a first annular flange 311 and a second annular flange 312. An annular groove 313 is formed between the first annular flange 311 and the second annular flange 312. The annular groove 313 serves to confine the buckle 212.

Referring to FIG. 1, in installation position, the first end portion 32 is near the buckle 212 and the second end portion 33 is near the handhold portion 113, while the injection needle 22 and the needle seat 21 of the injection needle 22 protrudes outwards from the first end portion 32. In this embodiment, the lower side of the injection cylinder 11 is (referring to FIG. 2) tightly engaged to the first flange 311 and the second flange 312 so that in installation position, the safety sleeve 3 is tightly engaged to the injection cylinder 11, and the first end portion 32 resists against the buckle 212 of the needle seat 21 for enhancing the structure of the needle seat 21 to prevent the needle seat 21 from separating with the injection cylinder 11. In detail, in FIG. 1, in the unused state of the injection needle apparatus with safe-protection function, the safety sleeve 3 is in the installation position, and the cover 4 covers upon the needle seat 21 for enclosing the injection needle 22. If it is necessary to use the injection needle apparatus with safe-protection function of the present invention, the cover 4 is removed.

Referring to FIG. 7, in use, the medical member removes the cover 4. By using the piston rod 12 to fill drug liquid into the injection cylinder 11, the safety sleeve 3 is moved to a protection position. In the protection position, the first end portion 33 is more near the head portion 112 to avoid the injection needle 22 to hurt medical members. In injection, the safety sleeve 3 is moved back to the installation position. Referring to FIG. 3, air could flow through the recesses 213a of the needle seat 21 so that when the safety sleeve 3 moves between the installation position and the protection position, it is not affected. Therefore, the movement is smooth.

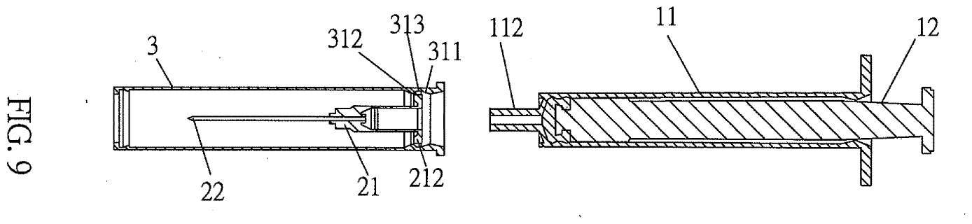

With reference to FIGS. 3, 8 and 9, when injection is finished, the safety sleeve 3 is pulled out to cause the second flange 312 goes through the buckle 212 and the buckle 212 is recessed in the annular groove 313 so that the annular groove 313 receives the buckle 212. Thereby, the safety sleeve 3 and the needle seat 21 can separate from the injection cylinder 11 integrally and the safety sleeve 3 covers the injection needle 22. In detail, when the second flange 312 moves over the buckle 212, the thin portions 214 causes that the buckle 212 to be easily deformed elastically so that the second flange 312 moves easily. After the second flange 312 moves through the buckle 212, the buckling blocks 213 is buckled in the annular grooves 313 so that the safety sleeve 3 is fixed to the needle seat 21 and the safety sleeve 3 covers completely the injection needle 22 so as to isolate the injection needle 22 from hurting anybody.

Next, the medical members pulls the needle seat 21 with the safety sleeve 3 to leave from the head portion 112, so that the needle seat 21 is separated from the injection cylinder 11 to be thrown away (see FIG. 9). Thus, the length of undesired injection needle apparatus with safe-protection function is reduced. Thereby, disposal of the injection cylinder 11 and the set of needle seat 21 and safety sleeve 3 can be done separately. If it is desired to inject drug liquid into a fixed injection device by using the injection cylinder 11, similarly, the needle seat 21 with the safety sleeve 3 is separated from the injection cylinder 11.

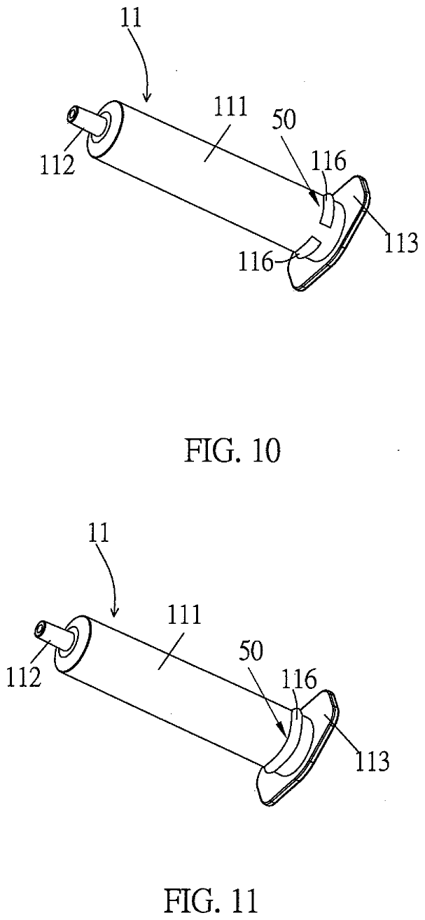

Referring to FIGS. 2, 10 and 13, in one embodiment of the present invention, a fixing structure 50 is formed at a lower end of the injection cylinder 11 near the handhold portion 113 and a rear end of the safety sleeve 3 so that when the safety sleeve 3 is pulled backwards to be near the handhold portion 113 of the injection cylinder 11, the safety sleeve 3 can be fixed as the position to prevent the safety sleeve 3 from moving with the injection needle 22 so as to interfere the operation of medical member.

Referring to FIG. 2, the first arrangement of the fixing structure 50 of the present invention is illustrated, in that a plurality of ribs 114 protruded from an outer periphery of the main body 111 near the handhold portion 113. In this embodiment, the ribs 114 is matched to the first annular flange 311 and the second annular flange 312 so that in the installation position, the safety sleeve 3 is tightly engaged with the injection cylinder 11 and the first end portion 32 resists against the buckle 212 for enhancing the fixness of the needle seat 21 to prevent the needle seat 21 from separating with the injection cylinder 11.

FIG. 11 shows the second arrangement of the fixing structure 50 of the present invention, in that an outer surface of the head portion 112 of the main body 111 is formed with a protrusion 116 the shape of which is corresponding to the annular groove 313 between the first annular flange 311 and the second annular flange 312 so that when the safety sleeve 3 retracts along the outer surface of the main body 111, the annular groove 313 will buckle the protrusion 116 so that the safety sleeve 3 is stopped on the main body 111. Therefore, in the installation position, the safety sleeve 3 is tightly engaged with the injection cylinder 11.

In this embodiment, as illustrated in FIG. 11, the protrusion 116 may be a complete annular protrusion enclosing the main body 111 or be a part of an annular protrusion enclosing a part of the main body 111, all these designs are within the scope of the present invention.

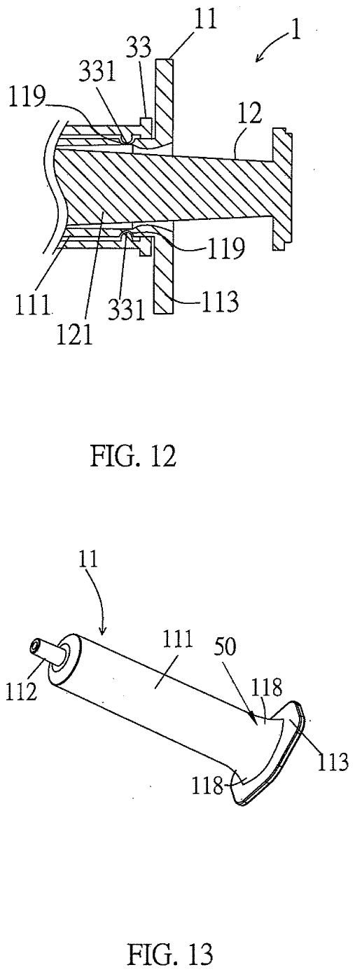

With reference to FIG. 12, the third arrangement of the fixing structure 50 of the present invention is illustrated. An outer surface of the main body 111 near the handhold portion 113 is formed with a concave portion 119; and corresponding portion of the safety sleeve 3 is formed with an annular protrusion 331 which can be buckled to the concave portion 119 of the present invention.

However, any structure with functions of buckling the safety sleeve 3 with the injection cylinder 11 are within the scope of the present invention.

FIG. 13 shows an application of the present invention about the fixing structure, wherein tightening engagement between the main body 111 and the safety sleeve 3 are used. In this embodiment, the outer surface of the main body 111 near the handhold portion 113 is formed with a thickening portion 118 so that when the safety sleeve 3 retracts along the outer surface of the main body 111, the safety sleeve 3 will gradually tightly engage with the outer surface of the thickening portion 118 of the handhold portion 113 so that the safety sleeve 3 is stopped on the main body 111 to achieve the requirement of safety operation.

In summary, the structure of the needle seat 21 and the safety sleeve 3 is simple and can be assembled and operated easily. Furthermore, the structure of needle seat 21 and safety sleeve 3 can be separated with the injection cylinder 11 so that undesired waste can be disposed easily. Furthermore, the present invention can be applied in injection of drug liquid.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An injection needle apparatus with safe-protection function, comprising:
an injection module (1) including an injection cylinder and a pullable piston rod installed within the injection cylinder; the injection cylinder including a main body, a head portion and a handhold portion; the head portion and the handhold portion being at two ends of the main body, respectively;
a needle module (2) including a needle seat and an injection needle; the needle seat having a seat body and a buckle; in installation state, the seat body covering the head portion and holding the injection needle; the buckle being integrally connected to the seat body at an end near the main body and around the seat body;
a safety sleeve (3) covering movably around the injection cylinder (11) between an installation position and a protection position; the safety sleeve having a sleeve body, a first end portion, and a second end portion; the first end portion and the second end portion being at two opposite ends of the sleeve body; wherein in the installing position, the first end portion is near the buckle and the second end portion is near the handhold portion; while in the protection position, the first end portion is protruded from the injection cylinder for covering the injection needle and the second end portion is near the head portion; and
an inner side of the sleeve body (31) near the second end portion being formed with a buckling portion; the buckling portion being used to buckle with the buckle (212) so that the safety sleeve (3) and the needle seat (21) are fixed together for separating from the injection cylinder and after separation, the safety sleeve covers the needle seat.

2. The injection needle apparatus with safe-protection function as claimed in claim 1, wherein a fixing structure (50) is formed at a lower end of the injection cylinder (11) near the handhold portion (113) and a rear end of the safety sleeve (3) so that when the safety sleeve retracts backwards to be near the handhold portion (113) of the injection cylinder (11), the safety sleeve can be fixed as the position to prevent the safety sleeve from moving with the injection needle.

3. The injection needle apparatus with safe-protection function as claimed in claim 1, wherein a tight buckling structure (52) is formed at the head portion (112) of the injection cylinder (11) and a rear end of the seat body (211) of the needle seat (21); therefore, the seat body (211) of the needle seat (21) can be tightly fixed to the head portion (112) of the needle seat (21) to prevent the needle seat (21) from separating with the head portion (112) of the injection cylinder (11).

4. The injection needle apparatus with safe-protection function as claimed in claim 2, wherein a tight buckling structure (52) is formed at the head portion (112) of the injection cylinder (11) and a rear end of the seat body (211) of the needle seat (21); therefore, the seat body (211) of the needle seat (21) can be tightly fixed to the head portion (112) of the needle seat (21) to prevent the needle seat (21) from separating with the head portion (112) of the injection cylinder (11).

5. The injection needle apparatus with safe-protection function as claimed in claim 1, wherein the buckling portion includes a first annular flange (311) and a second annular flange (312); an annular groove (313) being formed between the first annular flange and the annular second flange; the annular groove being used to confine the buckle.

6. The injection needle apparatus with safe-protection function as claimed in claim 4, wherein the buckling portion includes a first annular flange (311) and a second annular flange (312); an annular groove (313) being formed between the first annular flange and the annular second flange; the annular groove being used to confine the buckle.

7. The injection needle apparatus with safe-protection function as claimed in claim 1, wherein an outer surface of the head portion (112) of the injection cylinder (11) is formed with an outer thread (522), and an inner wall of the opening (215) of the needle seat (21) is formed with a respective inner thread (523); when assembling the needle seat into the head portion, the seat body can be threaded combined with the head portion.

8. The injection needle apparatus with safe-protection function as claimed in claim 4, wherein an outer surface of the head portion (112) of the injection cylinder (11) is formed with an outer thread (522), and an inner wall of the opening (215) of the needle seat (21) is formed with a respective inner thread (523); when assembling the needle seat into the head portion, the seat body can be threaded combined with the head portion.

9. The injection needle apparatus with safe-protection function as claimed in claim 1, wherein a plurality of ribs (114) protruded from an outer periphery of the main body (111) near the handhold portion (113); the ribs (114) is buckled with the buckling portion of the safety sleeve (3); so that in the installation position, the safety sleeve is tightly engaged with the injection cylinder.

10. The injection needle apparatus with safe-protection function as claimed in claim 1, wherein an outer surface of the head portion (112) of the main body (111) is formed with a protrusion (116) a shape of which is corresponding to the buckling portion of the safety sleeve (3); when the safety sleeve retracts along the outer surface of the main body (111), the protrusion (116) buckles with the buckling portion of the safety sleeve (3) so that so that the safety sleeve (3) is stopped on the main body (111).

11. The injection needle apparatus with safe-protection function as claimed in claim 1, wherein an outer surface of the head portion (112) of the main body (111) is formed with a protrusion (116) a shape of which is corresponding to the buckling portion of the safety sleeve (3); when the safety sleeve retracts along the outer surface of the main body (111), the protrusion (116) buckles with the buckling portion of the safety sleeve (3) so that so that the safety sleeve (3) is stopped on the main body (111).

12. The injection needle apparatus with safe-protection function as claimed in claim 11, wherein protrusion (116) is a complete annular protrusion enclosing the main body or be a part of an annular protrusion enclosing a part of the main body.

13. The injection needle apparatus with safe-protection function as claimed in claim 1, wherein an outer surface of the main body (111) near the handhold portion (113) is formed with a concave portion (119); and the buckling portion of the safety sleeve (3) is formed with an annular protrusion (331) which can be buckled to the concave portion (119) of the present invention.

14. The injection needle apparatus with safe-protection function as claimed in claim 1, wherein an outer surface of the main body (111) near the handhold portion (113) is formed with a thickening portion (118) so that when the safety sleeve (3) retracts along the outer surface of the main body (111), the safety sleeve (3) gradually tightly engages with the outer surface of the thickening portion (118) of the handhold portion (113) so that the safety sleeve (3) is stopped on the main body (111) to achieve the requirement of safety operation.

15. The injection needle apparatus with safe-protection function as claimed in claim 1, wherein the buckle is extended with a plurality of buckling blocks and a plurality of thin portions around an outer periphery thereof, the plurality of buckling blocks and the thin portions are alternatively arranged.

16. The injection needle apparatus with safe-protection function as claimed in claim 15 wherein each buckling block is formed with at least one recess.
